(19) United States
(12) Patent Application Publication
(10) Pub. No.: US 2006/0270926 A1
(43) Pub. Date: Nov. 30, 2006

(54) Methods and systems to segment central sulcus and Sylvian fissure

(76) Inventors: Qingmao Hu, Singapore (SG); Wieslaw Lucjan Nowinski, Singapore (SG); Zujun Hou, Singapore (SG); Wei Zuo, Singapore (SG); Wenlong Qian, Nottingham (GB)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/348,988

(22) Filed: Feb. 7, 2006

(30) Foreign Application Priority Data
Feb. 7, 2005 (SG) 200500680-4

(51) Int. Cl. A61B 5/05; G06K 9/00

(52) U.S. Cl. 600/407; 382/128

(57) ABSTRACT

Methods, computer systems, and computer program products for segmenting tissue based upon image data representing brain tissue are disclosed. In the method, image data representing brain tissue is reformatted to a predetermined spatial orientation. Spatial statistics relating to spatial features of the represented brain tissue are determined. The represented brain tissue is classified within categories of white matter, gray matter and cerebrospinal fluid. A region of interest in the represented brain tissue classified as cerebrospinal fluid is identified. The represented brain tissue proximate the identified region is segmented using a region-growing technique based on the identified region of interest.

### RELATED APPLICATION

The present patent application claims the benefit of an earlier filing date from Singapore Patent Application No. 200500680-4 filed on 7 Feb. 2005, which is herein incorporated by reference.

### TECHNICAL FIELD

The present invention relates generally to medical image processing and more particularly to imaging techniques that can be used for identifying and segmenting the central sulcus and Sylvian fissure in cranial images.

### BACKGROUND

Various authors have contributed to medical knowledge concerning automatic segmentation of sulci, but little literature exists concerning segmentation of the central sulcus (CS) and the Sylvian fissure (SF).

The central sulcus (CS) separates the parietal from frontal lobes. The CS starts in or near the superomedial border, slightly behind the midpoint between the frontal and occipital poles. The CS runs sinuously downwards and forwards for about 8 to 10 cm to end slightly above the posterior ramus of the lateral sulcus, from which it is always separated by an arched gyrus.

The Sylvian fissure (SF) is a cleft rising at a sharp angle, seen in both hemispheres of the brain, but more pronounced in the left. The cleft runs between Broca's area and Wernicke's area, both parts of the left hemisphere known to be implicated in language function. The SF plays very important role in parcellation, or division, of the cortical surface.

Both the CS and the SF are cerebrospinal fluid (CSF), though there are other CSFs as well. A selective survey of some of these techniques for segmentation of sulci is described.

Lohmann et al [Lohmann G., Cramon, D. Y. V., “Automatic labeling of the human cortical surface using sulcal basins”, Medical Image Analysis, 2000; 4: 179-88] propose segmenting the sulcal basins, as the union of the sulci and gray matter. Rettmann et al [Rettmann, M., Han X., Xu C., Prince J. L., “Automated sulcal segmentation using watersheds on the cortical surface”, NeuroImage, 2002; 15: 329-44] use watersheds to segment the sulcal regions, which are also taken to be the union of sulci and gray matter.

Mangin et al [Mangin, J. F., Frouin, V., Bloch, I., Regis, J., Lopez-Krahe, J. “From 3D magnetic resonance images to structural representations of the cortex topography using topology preserving deformations”, Journal of Mathematical Imaging and Vision, 1995; 5(4):297-318] used k-means to find the union of sulci and gray matter. Renault et al [Renault, C, Desvignes, M., Revenu, M., “3D curves tracking and its application to cortical sulci detection”, Proceedings of the 2000 IEEE International Conference on Image Processing, vol. 2: 491-4] propose a curve tracking technique for sulci detection.

The above authors are unable to report identifying any specific sulcus, due to the partial volume effect of the magnetic resonance images (MRIs).

Manceaux-Demiau et al [Manceaux-Demiau A, Bryan RN, Davatzikos C. A probabilistic ribbon model for shape analysis of the cerebral sulci: applications to the central sulcus. Journal of Computer Assisted Tomography 1998; 22(6): 962-71] proposed to quantify the central sulcus through probabilistic geometric features like curvature through training provided that the segmentation is available. Tao et al [Tao, X., Han, X., Rettmann, M., Prince J., Davatzikos, C., “Statistical study on cortical sulci of human brains”, Proceedings of Information Processing in Medical Imaging, 2001; 475-87] also used statistical features to quantify sulci.

A method for identifying and localizing the central sulcus from magnetic resonance images is not known. The above-mentioned techniques are unsuitable for segmenting the Sylvian fissure due to its complex anatomy. A need clearly exists for an improved technique of identifying the Sylvian fissure.

### SUMMARY

In accordance with an aspect of the invention, there is provided a method of segmenting tissue based upon image data representing brain tissue. The method comprises the steps of: reformatting image data representing brain tissue to a predetermined spatial orientation; determining spatial statistics relating to spatial features of the represented brain tissue; classifying the represented brain tissue within categories of white matter, gray matter and cerebrospinal fluid; identifying a region of interest in the represented brain tissue classified as cerebrospinal fluid; and segmenting the represented brain tissue proximate the identified region using a region-growing technique based on the identified region of interest.

The predetermined spatial orientation may define three orthogonal axes that are: (i) a normal vector to the midsagittal plane, (ii) a parallel vector to the line segment connecting the anterior commissure and the posterior commissure, and (iii) a vector product of the axes (i) and (ii).

The method may further comprise the step of identifying the central sulcal basin as a sulcal basin having maximum volume.

The method may further comprise the step of identifying the Sylvian sulcal basin as the sulcal basin from the coronal section passing the anterior commissure where the basin is connected to the skeleton of the sulcal basin.

The method may further comprise the step of reformatting the image data to a predetermined spatial scale.

The predetermined spatial scale may define a dimension of 1 mm along each of the orthogonal axes of each voxel.

The method may further comprise the step of preprocessing the image data to remove bone tissue from the image.

The tissue segmented based upon image data representing brain tissue may comprise the central sulcus and Sylvian fissure regions in an image of a brain.

In accordance with another aspect of the invention, there is provided a computer program product comprising a computer-readable medium having computer software recorded therein for segmenting tissue based upon image data representing brain tissue. The computer program product comprises: a computer software program code module for reformatting image data representing brain tissue to a predetermined spatial orientation; a computer software program code module for determining spatial statistics relating to spatial features of the represented brain tissue; a computer software program code module for classifying the represented brain tissue within categories of white matter, gray matter and cerebrospinal fluid; a computer software program code module for identifying a region of interest in the represented brain tissue classified as cerebrospinal fluid; and a computer software program code module for segmenting the represented brain tissue proximate the identified region using a region-growing technique based on the identified region of interest.

In accordance with yet another aspect of the invention, there is provided a computer system comprising computer software recorded on a computer-readable medium for segmenting tissue based upon image data representing brain tissue. The computer system further comprises a memory for storing at least a portion of the computer software read from the computer readable medium; a processor coupled to the memory for executing the computer software. The computer software comprises: a computer software program code module for reformatting image data representing brain tissue to a predetermined spatial orientation; a computer software program code module for determining spatial statistics relating to spatial features of the represented brain tissue; a computer software program code module for classifying the represented brain tissue within categories of white matter, gray matter and cerebrospinal fluid; a computer software program code module for identifying a region of interest in the represented brain tissue classified as cerebrospinal fluid; and a computer software program code module for segmenting the represented brain tissue proximate the identified region using a region-growing technique based on the identified region of interest.

### BRIEF DESCRIPTION OF THE DRAWINGS

A small number of embodiments are described hereinafter with reference to the drawings, in which:

FIG. 1 is a flow chart illustrating a process for segmenting the Sylvian fissure;

FIG. 2 is an image that indicates automatic seed selection, where the dotted line passes the AC and is orthogonal to the midsagittal line;

FIG. 3 is an image of restrictions to avoid region growing going far from Sylvian fissure;

FIG. 4A is an image of the skeleton of the Sylvian sulcal basin, and FIG. 4B is an image of the SF of a coronal slice;

FIGS. 5A to 5E are images illustrating respectively an original axial slice of a dataset (5A), the central sulcal basin of the same axial slice (5B), the skeleton (5C), the cerebrospinal fluid (5D), and the central sulcus of this axial slice (5E);

FIG. 6 is a schematic representation of a computer system suitable for performing the techniques described hereinafter; and

FIG. 7 is a flow chart illustrating a process for segmenting tissue based upon image data representing brain tissue.

### DETAILED DESCRIPTION

Methods, computer systems and computer program products for segmenting tissue based upon image data representing brain tissue are described hereinafter. In the following description, numerous specific details, including image processing operations such as thresholding and morphological operations, segmentation techniques, and the like are set forth. However, from this disclosure, it will be apparent to those skilled in the art that modifications and/or substitutions may be made without departing from the scope and spirit of the invention. In other circumstances, specific details may be omitted so as not to obscure the invention.

Some portions of the description that follows are explicitly or implicitly presented in terms of algorithms and representations of operations on data within a computer system or other device capable of performing computations. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical, or magnetic, or electromagnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that the above and similar terms are to be associated with the appropriate physical quantities and are merely convenient labels applied to these quantities. Unless specifically stated otherwise, and as apparent from the following, it will be appreciated that throughout the present specification, discussions utilizing terms such as “reformatting”, “reading”, “sending”, “determining”, “classifying”, “identifying” “segmenting”, “preprocessing”, “performing” or the like, refer to the action and processes of a computer system, or similar electronic device, that manipulates and transforms data represented as physical (electronic) quantities within the registers and memories of the computer system into other data similarly represented as physical quantities within the computer system memories or registers or other such information storage, transmission or display devices.

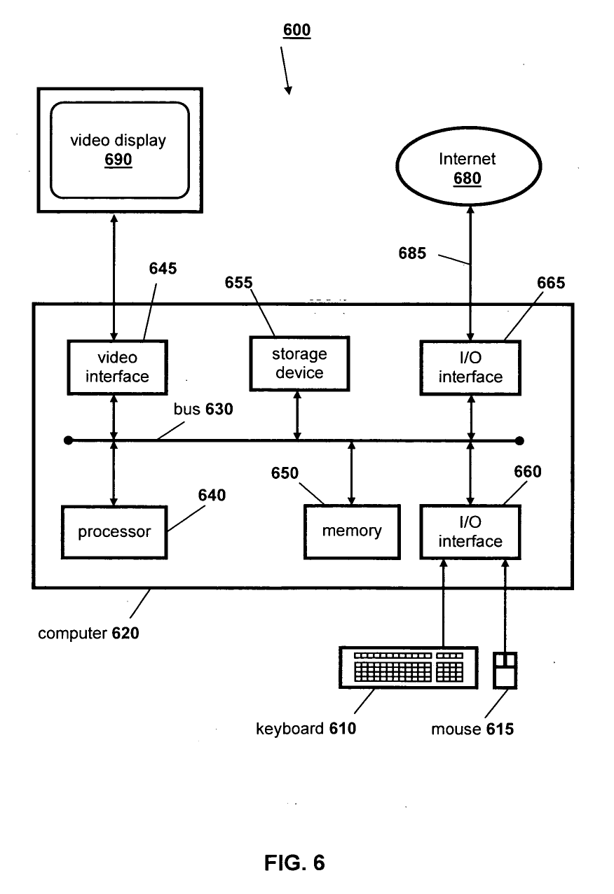

The present specification also discloses a computer system for performing the operations of the methods. Such an apparatus may be specially constructed for the required purposes, or may include a general-purpose computer or other device selectively activated or reconfigured by a computer program stored in the computer. The algorithms and displays presented herein are not inherently related to any particular computer or other apparatus. Various general-purpose machines may be used with programs in accordance with the teachings herein. Alternatively, the construction of more specialized apparatus to perform the required method steps may be appropriate. The structure of a conventional general-purpose computer is depicted in FIG. 6 and described in greater detail hereinafter.

In addition, the embodiments of the invention also disclose a computer program(s) or computer software, in that it would be apparent to the person skilled in the art that the individual steps of the methods described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing the spirit or scope of the invention. Furthermore one or more of the steps of the computer program may be performed in parallel rather than sequentially.

Such a computer program may be stored on any computer readable medium. The computer readable medium may include storage devices such as magnetic or optical disks, memory chips, or other storage devices suitable for interfacing with a general-purpose computer. The computer readable medium may also include a hard-wired medium such as exemplified in the Internet system, or wireless medium such as exemplified in a mobile telephone system or wireless local or wide area network. The computer program when loaded and executed on such a general-purpose computer effectively results in an apparatus or system that implements the steps of the method.

The method(s) may comprise a particular control flow. However, there are many other variants of the disclosed method(s) which use different control flows without departing the spirit or scope of the invention. Furthermore one or more of the steps of the disclosed method(s) may be performed in parallel rather than sequentially.

The methods may be implemented in modules. A module, and in particular its functionality, can be implemented in either hardware or software. In the software sense, a module is a process, program, or portion thereof that usually performs a particular function or related functions. Such software may be implemented in C, C++, JAVA, JAVA BEANS, Fortran, or a combination thereof, for example, but may be implemented in any of a number of other programming languages/systems, or combinations thereof. In the hardware sense, a module is a functional hardware unit designed for use with other components or modules. For example, a module may be implemented using discrete electronic components, or it may form at least a portion of an entire electronic circuit such as a Field Programmable Gate Arrays (FPGA), Application Specific Integrated Circuit (ASIC), and the like. A physical implementation may also comprise configuration data for a FPGA, or a layout for an ASIC, for example. Still further, the description of a physical implementation may be in EDIF netlisting language, structural VHDL, structural Verilog, or the like. Numerous other possibilities exist. Those skilled in the art will appreciate that the system may also be implemented as a combination of hardware and software modules.

Automatic identification and localization of the central sulcus (CS) and the Sylvian fissure (SF) regions may be performed for cranial magnetic resonance imaging (MRI) applications. Volumetric data is reformatted with respect to the midsagittal plane (MSP) and the anterior commissure (AC) and posterior commissure (PC). The central sulcal basin is the sulcal basin with the maximum volume. The Sylvian sulcal basin is identified from the Y-shaped sulcal basin in the coronal slice passing through the AC. The sulcus/fissure is the union of the skeleton of the corresponding basin and sulcus connected to the skeleton.

I. Overview

Currently, automated techniques for identifying and localizing the central sulcus (CS) and the Sylvian fissure (SF) are unknown. One possible technique is to identify and localize the CS and SF through manual editing of the segmented sulcal basins (a union of the cerebrospinal fluid and the gray matter), but this procedure is tedious and prone to error.

The CS and SF are both cerebrospinal fluid (CSF) as noted above. Both the SF and the CS are important in quantifying the cortical surface. These regions share similar spatial features. Both display folds having a long and narrow structure, and are consequently affected by the partial volume effect. In acquired MRI images, the SF and the CS are represented as close in intensity, and both are classified as CSF. These similarities mean that the approach to segmentation of both the SF and the CS shares some common characteristics.

Both the CS and the SF suffer problems relating to the partial volume effect and noise when segmentation is attempted. Existing techniques are thought to fail when applied to segmentation of the CS and SF, as existing techniques consider CSF as a whole, and are unable to identify the CS and SF, let alone separately segment each component. Further, the segmentation of CSF may not be complete due to the partial volume effect and the lack of any anatomical guidance, namely anatomical knowledge that can be used as a reference to guide the approach to segmentation.

The technique described herein, by contrast, use features (both anatomical and gray-level) to identify the CS and SF, and accurate segmentation is able to be subsequently performed. That is, accurate segmentation is performed only when one is sure that the CSF relates either to the CS or the SF. As well as segmentation, the midsagittal plane (MSP) and the landmarks such as the anterior commissure (AC) and the posterior commissure (PC) are used to automatically identify the position of the SF or the CS in MR images.

Segmentation methods for the CS and SF both involve the identification of the region of interest (ROI) relative to the MSP and the landmarks. Also, both procedures segment the sulcal basins for further refinement. Both methods derive the skeleton of the sulcal basin previously obtained and determine those voxels that can be classified as CSF, and which represent tissue connected to the skeleton.

The techniques described herein can automatically identify and localize the CS and the SF (the cerebrospinal fluid) for cranial MRI applications. The volumetric data is reformatted with respect to the MSP and the AC and PC. The central sulcal basin is the sulcal basin with the maximum volume. The Sylvian sulcal basin is identified from the Y-shaped sulcal basin in the coronal slice, or coronal section, passing through the AC. The sulcus/fissure is the union of the skeleton of the corresponding basin and sulcus/fissure connected to the skeleton.

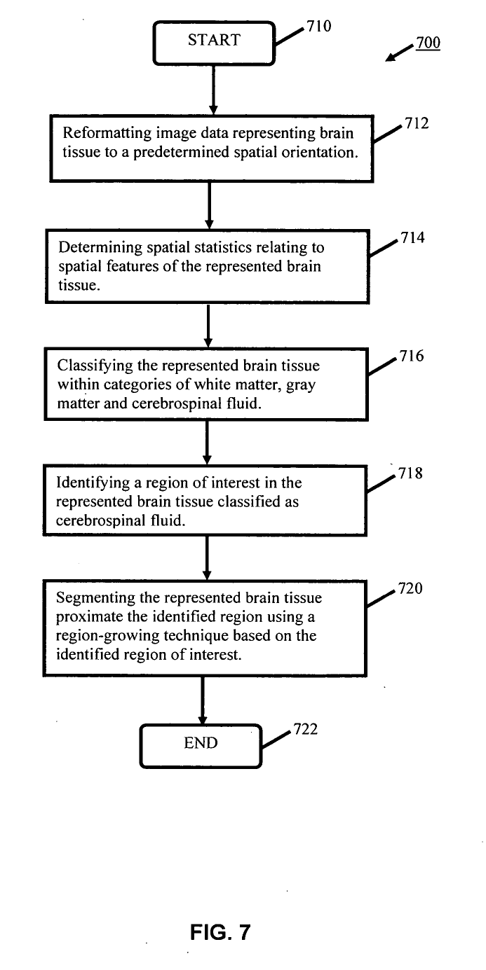

FIG. 7 is a high-level flow diagram illustrating a process 700 of segmenting tissue based upon image data representing brain tissue in accordance with an embodiment of the invention. Processing starts in step 710. In step 712, image data representing brain tissue is reformatted to a predetermined spatial orientation. In step 714, spatial statistics relating to spatial features of the represented brain tissue are determined. In step 716, the represented brain tissue is classified within categories of white matter, gray matter and cerebrospinal fluid. In step 718, a region of interest in the represented brain tissue classified as cerebrospinal fluid is identified. In step 720, the represented brain tissue proximate the identified region is segmented using a region-growing technique based on the identified region of interest. Processing terminates in step 722. Preferably, the tissue segmented based upon image data representing brain tissue comprises the central sulcus and Sylvian fissure regions in an image of a brain. Further details of this method are described hereinafter.

II. Process for Segmenting the Sylvian Fissure

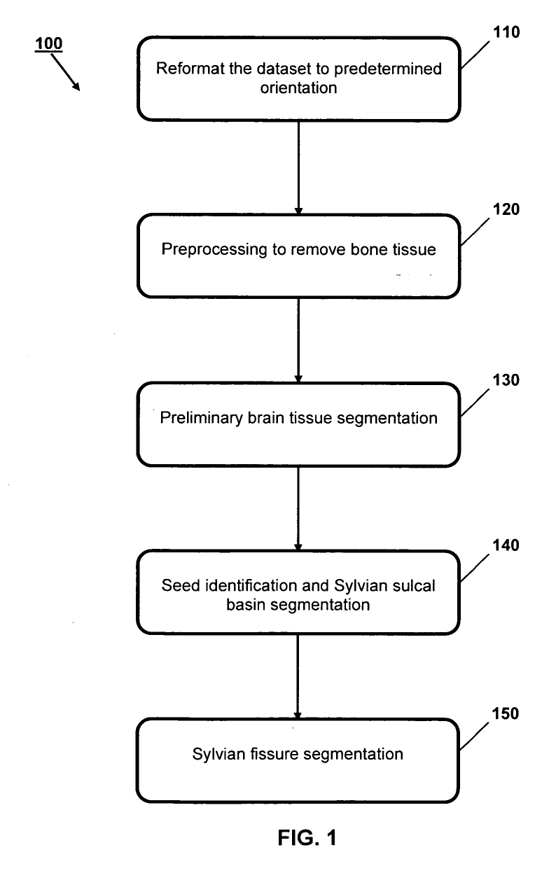

FIG. 1 illustrates the process 100 for segmenting the Sylvian fissure in accordance with an embodiment of the invention. The process 100 comprises the following steps. In step 110, the dataset is reformatted to a predetermined orientation. In step 120, preprocessing is performed to remove bone tissue. In step 130, preliminary brain tissue segmentation is carried out. In step 140, seed identification and sulcal basin segmentation are performed. In step 150, Sylvian fissure (CS/SF) segmentation is performed. Each of these steps is described in further detail hereinafter.

Step 110 Reformatting

First, the midsagittal plane (MSP) and the three-dimensional (3D) co-ordinates of the anterior commissure (AC) and the posterior commissure (PC) for the input of a MRI volume are computed. The dataset is reformatted so that the following conditions (a) to (d) are met:
(a) the new X-axis has the same direction as the normal vector of the MSP;
(b) the new Y-axis has the same direction as the line connecting the AC and PC;
(c) the new Z-axis has the same direction as the vector product of the new X- and Y-axes; and
(d) the volume pixel (voxel) sizes in X-, Y-, and Z-axes are all 1 mm, and the gray level at each voxel can be calculated using linear interpolation from the gray levels of the original dataset.
   Step 120 Preprocessing

The bone tissue (i.e., the scalp/skull) is removed from the MR image using thresholding and morphological operations.

Step 130 Preliminary Brain Tissue Segmentation

Statistics relating to brain tissue (such as mean value and standard deviation) are obtained for use in other steps. The image derived as a result of step 120 is segmented using the fuzzy c-means (FCM) method for the SF segmentation, and using thresholding combined with morphological processing for CS segmentation.

For the segmentation of both SF and CS, the brain tissues are classified into cerebrospinal fluid (CSF), gray matter (GM) and white matter (WM). Information about both the CSF and GM is used to segment the sulcal basin in step 140. CSF information further used for final segmentation of SF and CS in step 150.

Step 140 Seed Identification and Sulcal Basin Segmentation

For SF segmentation, the step 140 starts from the coronal section containing the AC, because in this slice the SF section is well Y-shaped. Further, if one makes a line through and orthogonal to the mid-sagittal line, which could be taken as the intersection of the MSP and the current coronal slice, this line also goes through the SF as well.

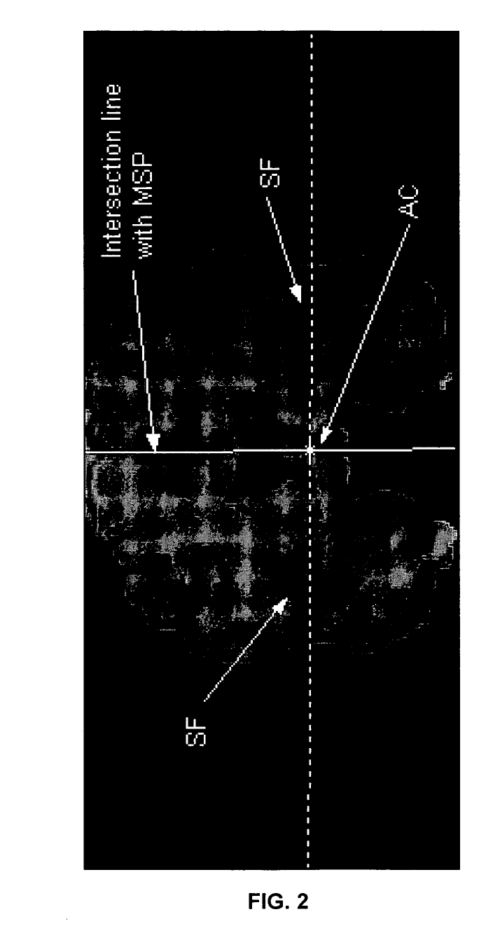

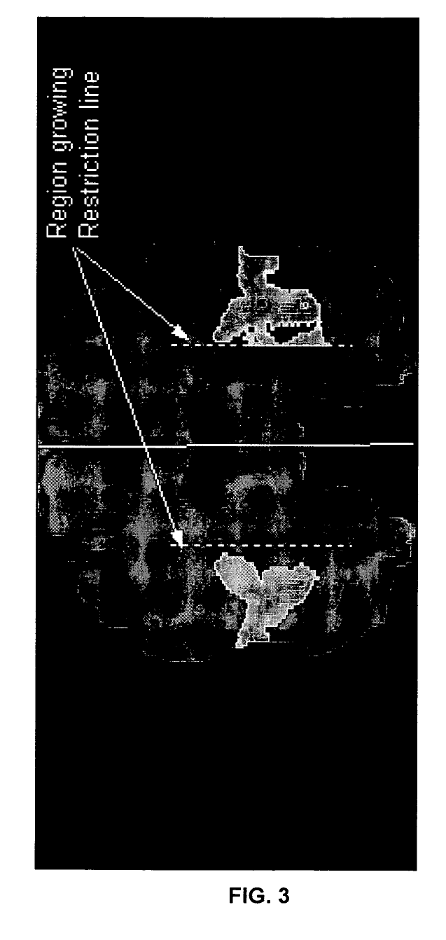

Two seeds in SF can be selected from the dotted line in FIG. 2. The region of interest (ROI) is determined as follows. The right boundary is defined by a line which is parallel to the mid-sagittal line. The distance of the right boundary to the mid-sagittal line is equal to the distance to the left extent of brain mask, as indicated in FIG. 3. The same restriction is applied to the right SF in FIG. 2 as well. This is based on the anatomical knowledge of interior extension of the SF.

After the region is extracted in the coronal section containing the AC, the sulcal basin of GM and CSF in the SF is extracted by the region growing technique, which uses the CSF and GM information from the preliminary segmentation of step 130. Seed points for other slices are automatically selected based on an extracted 2D region for its neighbouring slices.

For CS segmentation, step 140 includes determining the ROI and reference slice, by 3D region growing, and taking the sulcal basin with the maximum volume as the central sulcal basin.

The 3D ROI has the following extension: X from the leftmost cortical point to the rightmost cortical point of the axial slice passing through the AC and PC, Y from y_{AC} to y_{PC}+30 mm (where AC and PC's co-ordinates are denoted as (x_{AC}, y_{AC}, z_{AC}) and (x_{PC}, y_{PC}, z_{PC})), Z from the first axial slice with cortex (denote the z coordinate of this axial slice as z₀) to z_{AC}. The z co-ordinate of the reference axial slice is ⅚×z_{AC}+⅙×z₀.

Assume the MSP equation is x=x_{MSP}, where x_{MSP} is a positive constant. In the reference axial slice, 2 lines x₁=x_{MSP}+15 and x₂=x_{MPS}−15 are drawn. There are several intersected CSF/GM voxels with these 2 lines between (and near) AC and PC. Set these CSF/GM voxels as the seeds for 3D region growing to find their sulcal basin volumes. The sulcal basin with the maximum volume is the central sulcal basin.

Step 150 CS or SF Segmentation

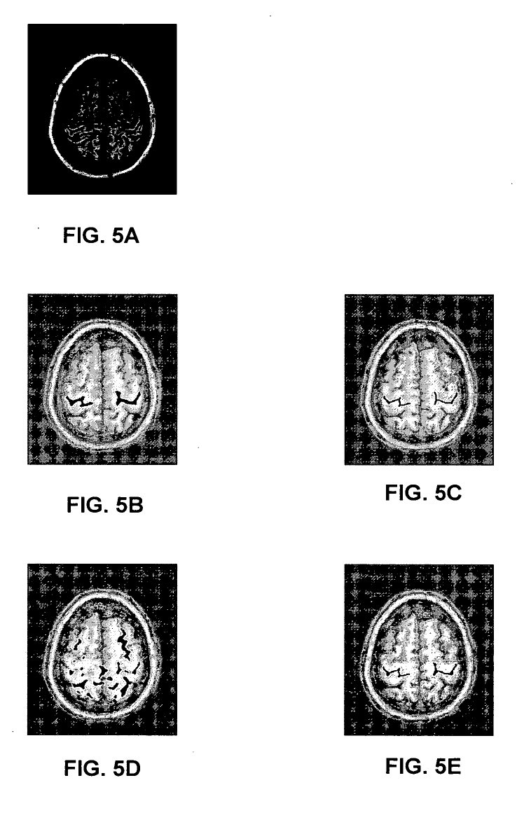

The skeleton of the central/Sylvian sulcal basin is obtained through two-dimensional (2D) skeletonization of the central/sylvian sulcal basin of the axial/coronal slice within the ROI. FIG. 5A illustrates an original axial slice of a dataset. This skeletonization can be achieved through any existing 2D skeletonization method. FIG. 5C shows the skeleton of the FIG. 5B.

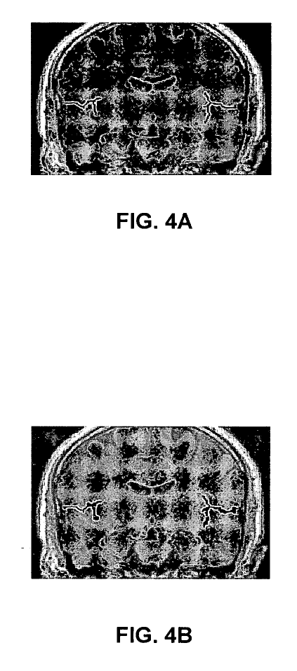

The CSF of the central sulcal basin is calculated through thresholding while the CSF of the sylvian sulcal basin is located through voxel membership function with the consideration of the almost constant thickness (there is variation across different regions) of cortices. FIG. 4A shows the skeleton of the sulcal basin in the SF. The SF is the union of the skeleton and the CSF voxels, which are from the preliminary segmentation of step 130, connected to the skeleton of the sulcal basin, as indicated in FIG. 4B.

FIG. 5D shows the CSF of the axial slice. The CS is the union of the skeleton and the CSF voxels connected to the skeleton of the central sulcal basin. FIG. 5E illustrates the central sulcus of this axial slice.

III. Computer System Implementation

The processes of FIGS. 1 to 5 and 7 may be implemented as software, such as an application program executing within the computer system or a handheld device. In particular, the steps of the method for segmenting tissue based upon image data representing brain tissue processing are effected, at least in part, by instructions in the software that are carried out by the computer. The instructions may be formed as one or more modules, each for performing one or more particular tasks. The software may be stored in a computer readable medium, including the storage devices described below, for example. The software is loaded into the computer from the computer readable medium, and then executed by the computer. A computer readable medium having such software or computer program recorded on it is a computer program product.

The term “computer readable medium” as used herein refers to any storage or transmission medium that participates in providing instructions and/or data to the computer system for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external of the computer module. Examples of transmission media include radio or infra-red transmission channels as well as a network connection to another computer or networked device, and the Internet or Intranets including e-mail transmissions and information recorded on Websites and the like.

FIG. 6 is a schematic representation of a computer system 600 of a type that is suitable for executing computer software for medical image processing as described herein. Computer software executes under a suitable operating system installed on the computer system 600, and may be thought of as comprising various software code means for achieving particular steps.

The components of the computer system 600 include a computer 620, a keyboard 610 and mouse 615, and a video display 690. The computer 620 includes a processor 640, a memory 650, input/output (I/O) interfaces 660, 665, a video interface 645, and a storage device 655.

The processor 640 is a central processing unit (CPU) that executes the operating system and the computer software executing under the operating system. The memory 650 includes random access memory (RAM) and read-only memory (ROM), and is used under direction of the processor 640.

The video interface 645 is connected to video display 690 and provides video signals for display on the video display 690. User input to operate the computer 620 is provided from the keyboard 610 and mouse 615. The storage device 655 can include a disk drive or any other suitable storage medium.

Each of the components of the computer 620 is connected to an internal bus 630 that includes data, address, and control buses, to allow components of the computer 620 to communicate with each other via the bus 630.

The computer system 600 can be connected to one or more other similar computers via a input/output (I/O) interface 665 using a communication channel 685 to a network, represented as the Internet 680.

The computer software may be recorded on a portable storage medium, in which case, the computer software program is accessed by the computer system 600 from the storage device 655. Alternatively, the computer software can be accessed directly from the Internet 680 by the computer 620. In either case, a user can interact with the computer system 600 using the keyboard 610 and mouse 615 to operate the programmed computer software executing on the computer 620.

Other configurations or types of computer systems can be equally well used to execute computer software that assists in implementing the techniques described herein.

In the foregoing manner, methods, computer systems and computer program products for segmenting tissue based upon image data representing brain tissue have been described. Various alterations, substitutions and modifications can be made to the techniques and arrangements described herein, as would be apparent to one skilled in the relevant art in the light of this disclosure, without departing from the scope and spirit of the invention.

We claim:

1. A method of segmenting tissue based upon image data representing brain tissue, said method comprising the steps of:
reformatting image data representing brain tissue to a predetermined spatial orientation;
determining spatial statistics relating to spatial features of the represented brain tissue;
classifying the represented brain tissue within categories of white matter, gray matter and cerebrospinal fluid;
identifying a region of interest in the represented brain tissue classified as cerebrospinal fluid; and
segmenting the represented brain tissue proximate the identified region using a region-growing technique based on the identified region of interest.

2. The method as claimed in claim 1, wherein the predetermined spatial orientation defines three orthogonal axes that are (i) a normal vector to the midsagittal plane, (ii) a parallel vector to the line segment connecting the anterior commissure and the posterior commissure, and (iii) a vector product of the axes (i) and (ii).

3. The method as claimed in claim 1, further comprising the step of identifying the central sulcal basin as a sulcal basin having maximum volume.

4. The method as claimed in claim 1, further comprising the step of identifying the Sylvian sulcal basin as the sulcal basin from the coronal section passing through the anterior commissure where the basin is connected to the skeleton of the sulcal basin.

5. The method as claimed in claim 1, further comprising the step of reformatting the image data to a predetermined spatial scale.

6. The method as claimed in claim 1, wherein the predetermined spatial scale defines a dimension of 1 mm along each of the orthogonal axes of each voxel.

7. The method as claimed in claim 1, further comprising the step of preprocessing the image data to remove bone tissue from the image.

8. The method as claimed in claim 1, wherein said tissue segmented based upon image data representing brain tissue comprises the central sulcus and Sylvian fissure regions in an image of a brain.

9. A computer program product comprising a computer-readable medium having computer software recorded therein for segmenting tissue based upon image data representing brain tissue, said computer program product comprising:
computer software program code means for reformatting image data representing brain tissue to a predetermined spatial orientation;
computer software program code means for determining spatial statistics relating to spatial features of the represented brain tissue;
computer software program code means for classifying the represented brain tissue within categories of white matter, gray matter and cerebrospinal fluid;
computer software program code means for identifying a region of interest in the represented brain tissue classified as cerebrospinal fluid; and
computer software program code means for segmenting the represented brain tissue proximate the identified region using a region-growing technique based on the identified region of interest.

10. The computer program product as claimed in claim 9, wherein the predetermined spatial orientation defines three orthogonal axes that are (i) a normal vector to the midsagittal plane, (ii) a parallel vector to the line segment connecting the anterior commissure and the posterior commissure, and (iii) a vector product of the axes (i) and (ii).

11. The computer program product as claimed in claim 9, further comprising computer software program code means for identifying the central sulcal basin as a sulcal basin having maximum volume.

12. The computer program product as claimed in claim 9, further comprising computer software program code means for identifying the Sylvian sulcal basin as the sulcal basin from the coronal section passing the anterior commissure where the basin is connected to the skeleton of the sulcal basin.

13. The computer program product as claimed in claim 9, further comprising computer software program code means for reformatting the image data to a predetermined spatial scale.

14. The computer program product as claimed in claim 9, wherein the predetermined spatial scale defines a dimension of 1 mm along each of the orthogonal axes of each voxel.

15. The computer program product as claimed in claim 9, further comprising computer software program code means for preprocessing the image data to remove bone tissue from the image.

16. The computer program product as claimed in claim 9, wherein said tissue segmented based upon image data representing brain tissue comprises the central sulcus and Sylvian fissure regions in an image of a brain.

17. A computer system comprising computer software recorded on a computer-readable medium for segmenting tissue based upon image data representing brain tissue, said computer system further comprising:
a memory for storing at least a portion of said computer software read from said computer readable medium;
a processor coupled to said memory for executing said computer software, comprising:
computer software program code means for reformatting image data representing brain tissue to a predetermined spatial orientation;
computer software program code means for determining spatial statistics relating to spatial features of the represented brain tissue;
computer software program code means for classifying the represented brain tissue within categories of white matter, gray matter and cerebrospinal fluid;
computer software program code means for identifying a region of interest in the represented brain tissue classified as cerebrospinal fluid; and
computer software program code means for segmenting the represented brain tissue proximate the identified region using a region-growing technique based on the identified region of interest.

18. The computer system as claimed in claim 17, wherein the predetermined spatial orientation defines three orthogonal axes that are (i) a normal vector to the midsagittal plane, (ii) a parallel vector to the line segment connecting the anterior commissure and the posterior commissure, and (iii) a vector product of the axes (i) and (ii).

19. The computer system as claimed in claim 17, further comprising computer software program code means for identifying the central sulcal basin as a sulcal basin having maximum volume.

20. The computer system as claimed in claim 17, further comprising computer software program code means for identifying the Sylvian sulcal basin as the sulcal basin from the coronal section passing the anterior commissure where the basin is connected to the skeleton of the sulcal basin.

21. The computer system as claimed in claim 17, further comprising computer software program code means for reformatting the image data to a predetermined spatial scale.

22. The computer system as claimed in claim 17, wherein the predetermined spatial scale defines a dimension of 1 mm along each of the orthogonal axes of each voxel.

23. The computer system as claimed in claim 17, further comprising computer software program code means for preprocessing the image data to remove bone tissue from the image.

24. The computer system as claimed in claim 17, wherein said tissue segmented based upon image data representing brain tissue comprises the central sulcus and Sylvian fissure regions in an image of a brain.
